(19) United States
(12) Patent Application Publication
(10) Pub. No.: US 2007/0054269 A1
(43) Pub. Date: Mar. 8, 2007

(54) Molecular cardiotoxicology modeling

(76) Inventors: Donna L. Mendrick, Gaithersburg, MD (US); Mark W. Porter, Gaithersburg, MD (US); Kory R. Johnson, Gaithersburg, MD (US); Brandon Higgs, Gaithersburg, MD (US); Arthur L. Castle, Gaithersburg, MD (US); Michael Elashoff, Gaithersburg, MD (US)

Correspondence Address: COOLEY GODWARD LLP, THE BROWN BUILDING - 875 15TH STREET, NW, SUITE 800, WASHINGTON, DC, 20005-2221, US

(21) Appl. No.: 10/541,937

(22) PCT Filed: Jan. 8, 2004
(86) PCT No.: PCT/US04/00240

§ 371 Date: Jul. 10, 2006

Related U.S. Application Data
(63) Continuation-in-part of application No. 10/338,044, filed on Jan. 8, 2003.
Application No. 10/338,044 is a continuation-in-part of application No. 10/191,803, filed on Jul. 10, 2002.
(60) Provisional application No. 60/303,819, filed on Jul. 10, 2001.
(60) Provisional application No. 60/305,623, filed on Jul. 17, 2001.
(60) Provisional application No. 60/369,351, filed on Apr. 3, 2002.
(60) Provisional application No. 60/377,611, filed on May 6, 2002.

(51) Int. Cl. C12Q 1/68; G06F 19/00

(52) U.S. Cl. 435/6; 702/20

(57) ABSTRACT

The present invention is based on the elucidation of the global changes in gene expression and the identification of toxicity markers in tissues or cells exposed to a known cardiotoxin. The genes may be used as toxicity markers in drug screening and toxicity assays. The invention includes a database of genes characterized by toxin-induced differential expression that is designed for use with microarrays and other solid-phase probes.

### RELATED APPLICATIONS

This application claims priority to U.S. application Ser. No. 10/338,044, filed Jan. 8, 2003, which is a continuation-in-part of U.S. application Ser. No. 10/191,803, filed Jun. 10, 2002, which claims priority to U.S. Provisional Application 60/303,819 filed on Jul. 10, 2001; 60/305,623 filed on Jul. 17, 2001; 60/369,351 filed on Apr. 3, 2002; and 60/377,611 filed on May 6, 2002, all of which are herein incorporated by reference in their entirety.

This application is also related to U.S. application Ser. Nos. 09/917,800; 10/060,087; 10/152,319; and 10/301,856, all of which are also herein incorporated by reference in their entirety.

### SEQUENCE LISTING SUBMISSION ON COMPACT DISC

The Sequence Listing submitted concurrently herewith on compact disc under Section 801(a)(i) and under 37 C.F.R. §§ 1.821(c) and 1.821(e) is herein incorporated by reference in its entirety. Four copies of the Sequence Listing, one on each of four compact discs are provided. Copy 1, Copy 2 and Copy 3 are identical. Copies 1, 2 and 3 are also identical to the CRF. Each electronic copy of the Sequence Listing was created on Jan. 7, 2004 with a file size of 3952 KB. The file names are as follows: Copy 1-g1 5090 01 wo.txt; Copy 2-g1 5090 01 wo.txt; Copy 3-g1 5090 01 wo.txt; and CRF-g1 5090 01 wo.txt.

### BACKGROUND OF THE INVENTION

The need for methods of assessing the toxic impact of a compound, pharmaceutical agent or environmental pollutant on a cell or living organism has led to the development of procedures which utilize living organisms as biological monitors. The simplest and most convenient of these systems utilize unicellular microorganisms such as yeast and bacteria, since they are the most easily maintained and manipulated. In addition, unicellular screening systems often use easily detectable changes in phenotype to monitor the effect of test compounds on the cell. Unicellular organisms, however, are inadequate models for estimating the potential effects of many compounds on complex multicellular animals, in part because they do not have the ability to carry out biotransformations to the extent or at levels found in higher organisms.

The biotransformation of chemical compounds by multicellular organisms is a significant factor in determining the overall toxicity of agents to which they are exposed. Accordingly, multicellular screening systems may be preferred or required to detect the toxic effects of compounds. The use of multicellular organisms as toxicology screening tools has been significantly hampered, however, by the lack of convenient screening mechanisms or endpoints, such as those available in yeast or bacterial systems. In addition, previous attempts to produce toxicology prediction systems have failed to provide the necessary modeling data and statistical information to accurately predict toxic responses (e.g., WO 00/12760, WO 00/47761, WO 00/63435, WO 01/32928, and WO 01/38579).

### DESCRIPTION OF THE TABLES

Table 1 provides the GenBank Accession Number for each of the sequences of the invention (see www.ncbi.nlm.nih.gov/), as well as the corresponding SEQ ID NO. in the sequence listing filed with this application. The gene name and Unigene Cluster Title, if known, cardiotoxicity prediction model code and internal reference no. are also provided.

Table 2 lists and describes the metabolic pathways in which the genes of the invention are known to function.

Table 3 provides the LocusLink and Unigene names and descriptions for the human homologues of the genes listed in Tables 1 and 2.

Table 4 defines the model codes, each of which corresponds to a table in Tables 5A-5LL. Each of Tables 5A-5LL represents part of a cardiotoxicity prediction model and lists for each toxin, or class of toxins, the genes that are predictors of a toxic effect. For each gene listed, the mean and standard deviation for gene expression levels in Tox-Group and Non-tox Group samples, as well as the linear discriminant analysis score (LDA score), are indicated.

Table 5A lists the genes that predict a toxic effect in samples treated with adrenergic agonists.

Table 5B lists the toxicity prediction genes in samples treated with alkylating agents.

Table 5C lists the toxicity prediction genes in samples treated with adriamycin (120 and 168-hour time point data).

Table 5D lists the toxicity prediction genes in samples treated with adriamycin (6 and 24-hour time point data).

Table 5E lists the toxicity prediction genes in samples treated with amphotericin B.

Tables 5F and 5G list the toxicity prediction genes in an alternate model and in a core model, respectively, in samples treated with BI-QT, a proprietary heart and liver toxin.

Tables 5H and 5I list the toxicity prediction genes in an alternate model and in a core model, respectively, in samples treated with clenbuterol (24-hour time point data).

Tables 5J and 5K list the toxicity prediction genes in an alternate model and in a core model, respectively, in samples treated with clenbuterol (6-hour time point data).

Tables 5L and 5M list the toxicity prediction genes in an alternate model and in a core model, respectively, in samples treated with cyclophosphamide.

Tables 5N and 5O list the toxicity prediction genes in an alternate model and in a core model, respectively, in samples treated with epinephrine (24-hour time point data).

Tables 5P and 5Q list the toxicity prediction genes in an alternate model and in a core model, respectively, in samples treated with epinephrine (3 and 6-hour time point data).

Table 5R lists the toxicity prediction genes in samples treated with epirubicin.

Tables 5S and 5T list the toxicity prediction genes in an alternate model and in a core model, respectively, in samples treated with hydralazine.

Tables 5U and 5V list the toxicity prediction genes in an alternate model and in a core model, respectively, in samples treated with ifosfamide.

Tables 5W and 5X list the toxicity prediction genes in an alternate model and in a core model, respectively, in samples treated with isoproterenol (24-hour time point data).

Tables 5Y and 5Z list the toxicity prediction genes in an alternate model and in a core model, respectively, in samples treated with isoproterenol (3 and 6-hour time point data).

Tables 5AA and 5BB list the toxicity prediction genes in an alternate model and in a core model, respectively, in samples treated with minoxidil (3 and 6-hour time point data).

Tables 5CC and 5DD list the toxicity prediction genes in an alternate model and in a core model, respectively, in samples treated with norepinephrine (24-hour time point data).

Tables 5EE and 5FF list the toxicity prediction genes in an alternate model and in a core model, respectively, in samples treated with norepinephrine (3 and 6-hour time point data).

Tables 5GG (3-hour time point data) and 5HH (6 and 24-hour time point data) list the toxicity prediction genes in samples treated with phenylpropanolamine.

Table 5II lists the toxicity prediction genes in samples treated with rosiglitazone.

Tables 5JJ and 5KK list the toxicity prediction genes in a general model and in a general core model, respectively. The general model is produced by combining data from all the other models and includes, therefore, samples treated with various compounds and data taken at various time points. The general core model combines data from the core models produced using one toxin.

Table 5LL lists the toxicity prediction genes in samples treated with vasculature agents.

### SUMMARY OF THE INVENTION

The present invention is based, in part, on the elucidation of the global changes in gene expression in tissues or cells exposed to known toxins, in particular cardiotoxins, as compared to unexposed tissues or cells as well as the identification of individual genes that are differentially expressed upon toxin exposure.

In various aspects, the invention includes methods of predicting at least one toxic effect of a compound, predicting the progression of a toxic effect of a compound, and predicting the cardiotoxicity of a compound. The invention also includes methods of identifying agents that modulate the onset or progression of a toxic response. Also provided are methods of predicting the cellular pathways that a compound modulates in a cell. The invention also includes methods of identifying agents that modulate protein activities.

In a further aspect, the invention includes probes comprising sequences that specifically hybridize to genes in Tables 1-5LL. Also included are solid supports comprising at least two of the previously mentioned probes. The invention also includes a computer system that has a database containing information identifying the expression level. in a tissue or cell sample exposed to a cardiotoxin of a set of genes comprising at least two genes in Tables 1-5LL.

The invention further provides a core set of genes in Tables 5A-5LL from which probes can be made and attached to solid supports. These core genes serve as a preferred set of markers of cardiotoxicity and can be used with the methods of the invention to predict or monitor a toxic effect of a compound or to modulate the onset or progression of a toxic response.

### DETAILED DESCRIPTION

Many biological functions are accomplished by altering the expression of various genes through transcriptional (e.g. through control of initiation, provision of RNA precursors, RNA processing, etc.) and/or translational control. For example, fundamental biological processes such as cell cycle, cell differentiation and cell death are often characterized by the variations in the expression levels of groups of genes.

Changes in gene expression are also associated with the effects of various chemicals, drugs, toxins, pharmaceutical agents and pollutants on an organism or cell(s). For example, the lack of sufficient expression of functional tumor suppressor genes and/or the over-expression of oncogene/protooncogenes after exposure to an agent could lead to tumorgenesis or hyperplastic growth of cells (Marshall (1991), Cell 64: 313-326; Weinberg (1991), Science 254: 1138-1146). Thus, changes in the expression levels of particular genes (e.g. oncogenes or tumor suppressors) may serve as signposts for the presence and progression of toxicity or other cellular responses to exposure to a particular compound.

Monitoring changes in gene expression may also provide certain advantages during drug screening and development. Often drugs are screened for the ability to interact with a major target without regard to other effects the drugs have on cells. These cellular effects may cause toxicity in the whole animal, which prevents the development and clinical use of the potential drug.

The present inventors have examined tissue from animals exposed to known cardiotoxins which induce detrimental heart effects, to identify global changes in gene expression and individual changes in gene expression induced by these compounds. These global changes in gene expression, which can be detected by producing or obtaining gene expression profiles (an expression level of one or more genes), provide useful toxicity markers that can be used to monitor toxicity and/or toxicity progression by a test compound. Some of these markers may also be used to monitor or detect various disease or physiological states, disease progression, drug efficacy and drug metabolism.

Identification of Toxicity Markers

To evaluate and identify gene expression changes that are predictive of toxicity, studies using selected compounds with well characterized toxicity have been conducted by the present inventors to catalogue altered gene expression during exposure in vivo. In the present study, cyclophosphamide, ifosfamide, minoxidil, hydralazine, BI-QT, clenbuterol, isoproterenol, norepinephrine, epinephrine, adriamycin, amphotericin B, epirubicin, phenylpropanolamine, and rosiglitazone were selected as known cardiotoxins. Cisplatin, PAN, dopamine, acyclovir, carboplatin, etoposide, temozolomide, vancomycin and compound delivery vehicles were selected as negative controls.

Cyclophosphamide, an alkylating agent, is highly toxic to dividing cells and is commonly used in chemotherapy to treat non-Hodgkin's lymphomas, Burkitt's lymphoma and carcinomas of the lung, breast, and ovary (Goodman & Gilman's The Pharmacological Basis of Therapeutics 9th ed., p. 1234, 1237-1239, J. G. Hardman et al., Eds., McGraw Hill, New York, 1996). Additionally, cyclophosphamide is used as an immunosuppressive agent in bone marrow transplantation and following organ transplantation. Though cyclophosphamide is therapeutically useful, it is also associated with cardiotoxicity, nephrotoxicity, and hemorrhagic cystitis. Once in the liver, cyclophosphamide is hydroxylated by the cytochrome P450 mixed function oxidase system. The active metabolites, phosphoramide mustard and acrolein, cross-link DNA and cause growth arrest and cell death. Acrolein has been shown to decrease cellular glutathione levels (Dorr and Lagel (1994), Chem Biol Interact 93: 117-128).

The cardiotoxic effects of cyclophosphamide have been partially elucidated. One study analyzed plasma levels in 19 women with metastatic breast carcinoma who had been treated with cyclophosphamide, thiotepa, and carboplatin (Ayash et al. (1992), J Clin Oncol 10: 995-1000). Of the 19 women in the study, six developed moderate congestive heart failure. In another case study, a 10-year old boy, who had been treated with high-dose cyclophosphamide, developed cardiac arrhythmias and intractable hypotension (Tsai et al. (1990), Am J Pediatr Hematol Oncol 12: 472-476). The boy died 23 days after the transplantation.

Another clinical study examined the relationship between the amount of cyclophosphamide administered and the development of cardiotoxicity (Goldberg et al. (1986), Blood 68: 1114-1118). When the cyclophosphamide dosage was ≦1.55 g/m²/d, only 1 out of 32 patients had symptoms consistent with cyclophosphamide cardiotoxicity. Yet when the dosage was greater than 1.55 g/m²/d, 13 out of 52 patients were symptomatic. Six of the high-dose patients died of congestive heart failure.

In a related study, Braverman et al. compared the effects of once daily low-dose administration of cyclophosphamide (87+/−11 mg/kg) and twice-daily high-dose treatment (174+/−34 mg/kg) on bone marrow transplantation patients (Braverman et al. (1991), J Clin Oncol 9: 1215-1223). Within a week, the high-dose patients had an increase in left ventricular mass index. Out of five patients who developed clinical cardiotoxicity, four were in the high-dose group.

Ifosfamide, an oxazaphosphorine, is an analog of cyclophosphamide. Whereas cyclophosphamide has two chloroethyl groups on the exocyclic nitrogen, ifosfamide contains one chloroethyl group on the ring nitrogen and the other on the exocyclic nitrogen. Ifosfamide is a nitrogen mustard and alkylating agent, commonly used in chemotherapy to treat testicular, cervical, and lung cancer, as well as sarcomas and lymphomas. Like cyclophosphamide, it is activated in the liver by hydroxylation, but it reacts more slowly and produces more dechlorinated metabolites and chloroacetaldehyde. Comparatively higher doses of ifosfamide are required to match the efficacy of cyclophosphamide.

Alkylating agents can cross-link DNA, resulting in growth arrest and cell death. Despite its therapeutic value, ifosfamide is associated with nephrotoxicity (affecting the proximal and distal renal tubules), urotoxicity, venooclusive disease, myelosuppression, pulmonary fibrosis and central neurotoxicity (Goodman & Gilman's The Pharmacological Basis of Therapeutics 9^{th} ed. p. 1234-1240, J. G. Hardman et al., Eds., McGraw Hill, New York, 1996). Ifosfamide can also cause acute severe heart failure and malignant ventricular arrhythmia, which may be reversible. Death from cardiogenic shock has also been reported (Cecil Textbook of Medicine 20^{th} ed., Bennett et al. eds., p. 331, W.B. Saunders Co., Philadelphia, 1996).

Studies of patients with advanced or resistant lymphomas or carcinomas showed that high-dose ifosfamide treatment produced various symptoms of cardiac disease, including dyspnea, tachycardia, decreased left ventricular contractility and malignant ventricular arrhythmia (Quezado et al. (1993), Ann Intern Med 118: 31-36; Wilson et al. (1992), J Clin Oncol 19: 1712-1722. Other patient studies have noted that ifosfamide-induced cardiac toxicity may be asymptomatic, although it can be detected by electrocardiogram and should be monitored (Pai et al. (2000), Drug Saf 22:263-302).

Minoxidil is an antihypertensive medicinal agent used in the treatment of high blood pressure. It works by relaxing blood vessels so that blood may pass through them more easily, thereby lowering blood pressure. By applying minoxidil to the scalp, it has recently been shown to be effective at combating hair loss by stimulating hair growth. Once minoxidil is metabolized by hepatic sulfotransferase, it is converted to the active molecule minoxidil N—O sulfate (Goodman & Gilman's The Pharmacological Basis of Therapeutics 9^{th} ed., pp. 796-797, J. G. Hardman et al., Eds., McGraw Hill, New York, 1996). The active minoxidil sulfate stimulates the ATP-modulated potassium channel consequently causing hyperpolarization and relaxation of smooth muscle. Early studies on minoxidil demonstrated that following a single dose of the drug, patients suffering from left ventricular failure exhibited a slightly increased heart rate, a fall in the mean arterial pressure, a fall in the systemic vascular resistance, and a slight increase in cardiac index (Franciosa and Cohn (1981) Circulation 63: 652-657).

Some common side effects associated with minoxidil treatment are an increase in hair growth, weight gain, and a fast or irregular heartbeat. More serious side effects are numbness of the hands, feet, or face, chest pain, shortness of breath, and swelling of the feet or lower legs. Because of the risks of fluid retention and reflex cardiovascular effects, minoxidil is often given concomitantly with a diuretic and a sympatholytic drug.

While minioxidil is effective at lowering blood pressure, it does not lead to a regression of cardiac hypertrophy. To the contrary, minoxidil has been shown to cause cardiac enlargement when administered to normotensive animals (Moravec et al. (1994) J Pharmacol Exp Ther 269: 290-296). Moravec et al. examined normotensive rats that had developed myocardial hypertrophy following treatment with minoxidil. The authors found that minoxidil treatment led to enlargement of the left ventricle, right ventricle, and interventricular septum.

Another rat study investigated the age- and dose-dependency of minoxidil-induced cardiotoxicity (Herman et al. (1996) Toxicology 110: 71-83). Rats ranging in age from 3 months to 2 years were given varying amounts of minoxidil over the period of two days. The investigators observed interstitial hemorrhages at all dose levels, however the hemorrhages were more frequent and severe in the older animals. The 2 year old rats had vascular lesions composed of arteriolar damage and calcification.

Hydralazine, an antihypertensive drug, causes relaxation of arteriolar smooth muscle. Such vasodilation is linked to vigorous stimulation of the sympathetic nervous system, which in turn leads to increased heart rate and contractility, increased plasma renin activity, and fluid retention (Goodman & Gilman's The Pharmacological Basis of Therapeutics 9^{th} ed., p. 794, J. G. Hardman et al., Eds., McGraw Hill, New York, 1996). The increased renin activity leads to an increase in angiotensin II, which in turn causes stimulation of aldosterone and sodium reabsorption.

Hydralazine is used for the treatment of high blood pressure (hypertension) and for the treatment of pregnant women suffering from high blood pressure (pre-eclampsia or eclampsia). Some common side effects associated with hydralazine use are diarrhea, rapid heartbeat, headache, decreased appetite, and nausea. Hydralazine is often used concomitantly with drugs that inhibit sympathetic activity to combat the mild pulmonary hypertension that can be associated with hydralazine usage.

In one hydralazine study, rats were given one of five cardiotoxic compounds (isoproterenol, hydralazine, caffeine, cyclophosphamide, or adriamycin) by intravenous injection (Kemi et al. (1996), J Vet Med Sci 58: 699-702). At one hour and four hours post-dose, early focal myocardial lesions were observed histopathologically. Lesions were observed in the rats treated with hydralazine four hours post-dose. The lesions were found in the inner one third of the left ventricular walls including the papillary muscles.

Another study compared the effects of isoproterenol, hydralazine and minoxidil on young and mature rats (Hanton et al. (1991), Res Commun Chem Pathol Pharmacol 71: 231-234). Myocardial necrosis was observed in both age groups, but it was more severe in the mature rats. Hypotension and reflex tachycardia were also seen in the hydralazine-treated rats.

BI-QT has been shown to induce QC prolongation in dogs and liver alterations in rats. Over a four week period, dogs treated with BI-QT exhibited sedation, decreased body weight, increased liver weight, and slightly increased levels of AST, ALP, and BUN. After three months of treatment, the dogs exhibited signs of cardiovascular effects.

Clenbuterol, a β2 adrenergic agonist, can be used therapeutically as a bronchial dilator for asthmatics. It also has powerful muscle anabolic and lipolytic effects. It has been banned in the United States but continues to be used illegally by athletes to increase muscle growth. In a number of studies, rats treated with clenbuterol developed hypertrophy of the heart and latissimus dorsi muscle (Doheny et al. (1998), Amino Acids 15: 13-25; Murphy et al. (1999), Proc Soc Exp Biol Med 221: 184-187; Petrou et al. (1995), Circulation 92: II483-II489).

In one study, mares treated with therapeutic levels of clenbuterol were compared to mares that were exercised and mares in a control group (Sleeper et al. (2002), Med Sci Sports Exerc 34: 643-650). The clenbuterol-treated mares demonstrated significantly higher left ventricular internal dimension and interventricular septal wall thickness at end diastole. In addition, the clenbuterol-treated mares had significantly increased aortic root dimensions, which could lead to an increased chance of aortic rupture.

In another study, investigators reported a case of acute clenbuterol toxicity in a human (Hoffman et al. (2001), J Toxicol 39: 339-344). A 28-year old woman had ingested a small quantity of clenbuterol, and the patient developed sustained sinus tachycardia, hypokalemia, hypophosphatemia, and hypomagnesemia.

Catecholamines are neurotransmitters that are synthesized in the adrenal medulla and in the sympathetic nervous system. Epinephrine, norepinephrine, and isoproterenol are members of the catecholamine sympathomimetic amine family (Casarett & Doull's Toxicology, The Basic Science of Poisons 6^{th} ed., p. 618-619, C. D. Klaassen, Ed., McGraw Hill, New York, 2001). They are chemically similar by having an aromatic portion (catechol) to which is attached an amine, or nitrogen-containing group.

Isoproterenol, an antiarrhythmic agent, is used therapeutically as a bronchodilator for the treatment of asthma, chronic bronchitis, emphysema, and other lung diseases. Some side effects of usage are myocardial ischemia, arrhythmias, angina, hypertension, and tachycardia. As a β receptor agonist, isoproterenol exerts direct positive inotropic and chronotropic effects. Peripheral vascular resistance is decreased along with the pulse pressure and mean arterial pressure. However, the heart rate increases due to the decrease in the mean arterial pressure.

Norepinephrine, an α and β receptor agonist, is also known as noradrenaline. It is involved in behaviors such as attention and general arousal, stress, and mood states. By acting on β-1 receptors, it causes increased peripheral vascular resistance, pulse pressure and mean arterial pressure. Reflex bradycardia occurs due to the increase in mean arterial pressure. Some contraindications associated with norepinephrine usage are myocardial ischemia, premature ventricular contractions (PVCs), and ventricular tachycardia.

Epinephrine, a potent α and β adrenergic agonist, is used for treating bronchoconstriction and hypotension resulting from anaphylaxis as well as all forms of cardiac arrest. Injection of epinephrine leads to an increase in systolic pressure, irregular contractility, and heart rate. Some side effects associated with epinephrine usage are cardiac arrhythmias, particularly PVCs, ventricular tachycardia, renal vascular ischemia, increased myocardial oxygen requirements, and hypokalemia.

Anthracyclines are antineoplastic agents used commonly for the treatment of breast cancer, leukemias, and a variety of other solid tumors. However, the usefulness of the drugs are limited due dose-dependent cardiomyopathy and ECG changes (Casarett & Doull's Toxicology, The Basic Science of Poisons 6^{th ed}., p. 619, C. D. Klaassen, Ed., McGraw Hill, New York, 2001).

Adriamycin (doxorubicin) is a cytotoxic anthracycline antiobiotic that inhibits the action of topoisomerase II. It has a wide spectrum of antitumor activity, however dose-related cardiotoxicity is a major side effect. The toxic effects are most likely due to the generation of free radicals (DeAtley et al. (1999), Toxicology 134: 51-62). In one study, rats were given a dose of either adriamycin alone or a dose of adriamycin following a dose of captopril (al-Shabanah et al. (1998), Biochem Mol Biol Int 45: 419-427). Those rats that were only given adriamycin developed myocardial toxicity after 24 hours manifested biochemically by an elevation of serum enzymes such as aspartate transaminase, lactate dehydrogenase, and creatine phosphokinase. The rats that were pre-treated with captopril exhibited a significant reduction in serum enzyme levels as well as restoration of white blood cell counts.

Epirubicin is a semisynthetic derivative of daunorubicin, an anthracycline, approved for the treatment of breast cancer (Casarett & Doull's Toxicology The Basic Science of Poisons 6^{th} ed., p. 619, C. D. Klaassen, Ed., McGraw Hill, New York, 2001). Yet, it, too, may induce cardiotoxicity. In one observational study, 120 patients with advanced breast cancer were followed before, during, and after treatment with epirubicin (Jensen et al. (2002), Ann Oncol 13: 699-709). Approximately 59% of the patients experienced a 25% relative reduction in left ventricular ejection fraction three years after epirubicin treatment, and of these patients 20% had deteriorated into having congestive heart failure.

Amphotericin B is a polyene, antifungal antibiotic used to treat fungal infections. Its clinical utility is limited by its nephrotoxicity and cardiotoxicity. Amphotericin B may depress myocardial contractility by blocking activation of slow calcium channels and inhibiting the influx of sodium ions (Casarett & Doull's Toxicology, The Basic Science of Poisons 6^{th} ed., p. 621, C. D. Klaassen, Ed., McGraw Hill, New York, 2001). It has been shown to increase the permeability of the sarcolemmal membrane, and patients given amphotericin B have developed ventricular tachycardia and cardiac arrest. This drug has been shown to induce cardiac arrest in rats as well. In the current study, amphotericin B led to an increase in serum Troponin T levels and some early signs of cardiomyopathy within 24 hours of one intravenous bolus injection.

Phenylpropanolamine was used in over-the-counter decongestants until recently, but was withdrawn when its association with cardiac deaths became known. It is both a beta-1 and alpha adrenergic receptor agonist and has been shown to induce cardiotoxicity in rats. In one rat study, phenylpropanolamine was shown to cause myocardial contractile depression without altering global coronary artery blood flow (Zaloga et al. (2000), Crit Care Med 28: 3679-3683).

In another study, rats were given single intraperitoneal doses of 1, 2, 4, 8, 16, or 32 mg/kg of phenylpropanolamine (Pentel et al. (1987), Fundam Appl Toxicol 9: 167-172). The animals exhibited dose-dependent increased blood pressure and, following termination, myocardial necrosis.

Rosiglitazone (Avandia) is a thiazolidinedione medication used to treat Type 2 diabetes. It reduces plasma glucose levels and glucose production and increases glucose clearance (Wagstaff and Goa (2002), Drugs 62: 1805-1837). Some side effects associated with rosiglitazone treatment are fluid retention, congestive heart failure, and liver disease. In patients who have heart failure or use insulin, there is a potential for mild-to-moderate peripheral edema with rosiglitazone treatment. It has been shown that patients that do not have heart failure or use insulin can also develop moderate-to-severe edema while using rosiglitazone (Niemeyer and Janney (2002), Pharmacotherapy 22: 924-929).

Toxicity Prediction and Modeling

The genes, gene expression information (including Tox Group means and standard deviations, Nontox Group means and standard deviations, and LDA scores) and gene expression profiles, as well as the portfolios and subsets of the genes provided in Tables 1-5LL, such as the core toxicity markers in Tables 5A-5LL, may be used to predict at least one toxic effect, including the cardiotoxicity of a test or unknown compound. As used herein, at least one toxic effect includes, but is not limited to, a detrimental change in the physiological status of a cell or organism. The response may be, but is not required to be, associated with a particular pathology, such as tissue necrosis, myocarditis, arrhythmias, tachycardia, myocardial ischemia, angina, hypertension, hypotension, dyspnea, and cardiogenic shock. Accordingly, the toxic effect includes effects at the molecular and cellular level. Cardiotoxicity is an effect as used herein and includes but is not limited to the pathologies of tissue necrosis, myocarditis, arrhythmias, tachycardia, myocardial ischemia, angina, hypertension, hypotension, dyspnea, and cardiogenic shock. As used herein, a gene expression profile comprises any representation, quantitative or not, of the expression of at least one mRNA species in a cell sample or population and includes profiles made by various methods such as differential display, PCR, hybridization analysis, etc.

In general, assays to predict the toxicity or cardiotoxicity of a test agent (or compound or multi-component composition) comprise the steps of exposing a cell population to the test compound, assaying or measuring the level of relative or absolute gene expression of one or more of the genes in Tables 1-5LL and comparing the identified expression level(s) to the expression levels disclosed in the Tables and database(s) disclosed herein. Assays may include the measurement of the expression levels of about 2, 3, 4, 5, 6, 7, 8, 9, 10, 15, 20, 25, 30, 50, 75, 100, 200, 500, 1000 or more genes from Tables 1-5LL, or ranges of these numbers, such as about 2-10, about 10-20, about 20-50, about 50-100, about 100-200, about 200-500 or about 500-1000 genes from Tables 1-5LL to create multi-gene expression profiles. Assays for toxicity prediction may also include the measurement of nearly all the genes in Tables 1-5LL. “Nearly all” or “substantially all” the genes or gene information may be considered to mean at least 80%, preferably 85%, 90% or 95%, of the genes or information in any one of or all of Tables 1-5LL.

The genes, gene expression information and databases of the present invention may also be used to predict the absence of a toxic effect, or the non-toxicity of a test compound. Gene expression profiles of cell or tissue samples from subjects or samples exposed to the test compound are prepared or obtained and then compared to those stored in a database of the invention. If the test sample gene expression profiles correlate with gene expression profiles classified as Non-tox Group samples, the test compound may considered not to produce a toxic effect.

Further, the gene expression information and databases of the present invention may also be used to predict the dosage or level of exposure at which a particular test compound produces a toxic effect. Groups of human or animal subjects may be treated with varying dosages of a test compound for varying lengths of time, or cell or tissue samples may be taken from groups of human or animal subjects and treated with varying dosages of a test compound for varying lengths of time. Alternatively, human or animal cell cultures may be exposed to varying dosages of a test compound for varying lengths of time. Gene expression profiles may then be prepared or obtained from the set of samples treated with the test compound. These gene expression profiles may subsequently be compared to gene expression profiles stored in a database of the invention. In the sample set, the lowest concentration or dosage of the test compound that produces a gene expression profile that matches a gene expression profile indicating a toxic effect (corresponding to one or more Tox-Group samples in the database) may be determined. This concentration or dosage may be considered to be the threshold level at or above which a toxic response or effect may be predicted.

In the methods of the invention, the gene expression level for a gene or genes induced by the test agent, compound or compositions may be comparable to the levels found in the Tables or databases disclosed herein if the expression level varies within a factor of about 2, about 1.5 or about 1.0 fold. In some cases, the expression levels are comparable if the agent induces a change in the expression of a gene in the same direction (e.g., up or down) as a reference toxin.

The cell population that is exposed to the test agent, compound or composition may be exposed in vitro or in vivo. For instance, cultured or freshly isolated heart cells, in particular rat heart cells, may be exposed to the agent under standard laboratory and cell culture conditions. In another assay format, in vivo exposure may be accomplished by administration of the agent to a living animal, for instance a laboratory rat.

Procedures for designing and conducting toxicity tests in in vitro and in vivo systems are well known, and are described in many texts on the subject, such as Loomis et al., Loomis's Essentials of Toxicology 4th Ed., Academic Press, New York, 1996; Echobichon, The Basics of Toxicity Testing, CRC Press, Boca Raton, 1992; Frazier, editor, In Vitro Toxicity Testing, Marcel Dekker, New York, 1992; and the like.

In in vitro toxicity testing, two groups of test organisms are usually employed: one group serves as a control and the other group receives the test compound in a single dose (for acute toxicity tests) or a regimen of doses (for prolonged or chronic toxicity tests). Because, in some cases, the extraction of tissue as called for in the methods of the invention requires sacrificing the test animal, both the control group and the group receiving compound must be large enough to permit removal of animals for sampling tissues, if it is desired to observe the dynamics of gene expression through the duration of an experiment.

In setting up a toxicity study, extensive guidance is provided in the literature for selecting the appropriate test organism for the compound being tested, route of administration. dose ranges, and the like. Water or physiological saline (0.9% NaCl in water) is the solute of choice for the test compound since these solvents permit administration by a variety of routes. When this is not possible because of solubility limitations, vegetable oils such as corn oil or organic solvents such as propylene glycol may be used.

Regardless of the route of administration, the volume required to administer a given dose is limited by the size of the animal that is used. It is desirable to keep the volume of each dose uniform within and between groups of animals. When rats or mice are used, the volume administered by the oral route generally should not exceed about 0.005 ml per gram of animal. Even when aqueous or physiological saline solutions are used for parenteral injection, the volumes that are tolerated are limited, although such solutions are ordinarily thought of as being innocuous. The intravenous LD₅₀ of distilled water in the mouse is approximately 0.044 ml per gram and that of isotonic saline is 0.068 ml per gram of mouse. In some instances, the route of administration to the test animal should be the same as, or as similar as possible to, the route of administration of the compound to man for therapeutic purposes.

When a compound is to be administered by inhalation, special techniques for generating test atmospheres are necessary. The methods usually involve aerosolization or nebulization of fluids containing the compound. If the agent to be tested is a fluid that has an appreciable vapor pressure, it may be administered by passing air through the solution under controlled temperature conditions. Under these conditions, dose is estimated from the volume of air inhaled per unit time, the temperature of the solution, and the vapor pressure of the agent involved. Gases are metered from reservoirs. When particles of a solution are to be administered, unless the particle size is less than about 2 μm the particles will not reach the terminal alveolar sacs in the lungs. A variety of apparatuses and chambers are available to perform studies for detecting effects of irritant or other toxic endpoints when they are administered by inhalation. The preferred method of administering an agent to animals is via the oral route, either by intubation or by incorporating the agent in the feed.

When the agent is exposed to cells in vitro or in cell culture, the cell population to be exposed to the agent may be divided into two or more subpopulations, for instance, by dividing the population into two or more identical aliquots. In some preferred embodiments of the methods of the invention, the cells to be exposed to the agent are derived from heart tissue. For instance, cultured or freshly isolated rat heart cells may be used.

The methods of the invention may be used generally to predict at least one toxic response, and, as described in the Examples, may be used to predict the likelihood that a compound or test agent will induce various specific heart pathologies, such as tissue necrosis, myocarditis, arrhythmias, tachycardia, myocardial ischemia, angina, hypertension, hypotension, dyspnea, cardiogenic shock, or other pathologies associated with at least one of the toxins herein described. The methods of the invention may also be used to determine the similarity of a toxic response to one or more individual compounds. In addition, the methods of the invention may be used to predict or elucidate the potential cellular pathways influenced, induced or modulated by the compound or test agent due to the similarity of the expression profile compared to the profile induced by a known toxin (see Tables 5-5LL).

Diagnostic Uses for the Toxicity Markers

As described above, the genes and gene expression information or portfolios of the genes with their expression information as provided in Tables 1-5LL may be used as diagnostic markers for the prediction or identification of the physiological state of a tissue or cell sample that has been exposed to a compound or to identify or predict the toxic effects of a compound or agent. For instance, a tissue sample such as a sample of peripheral blood cells or some other easily obtainable tissue sample may be assayed by any of the methods described above, and the expression levels from a gene or genes from Tables 5-5LL may be compared to the expression levels found in tissues or cells exposed to the toxins described herein. These methods may result in the diagnosis of a physiological state in the cell, may be used to diagnose toxin exposure or may be used to identify the potential toxicity of a compound, for instance a new or unknown compound or agent that the subject has been exposed to. The comparison of expression data, as well as available sequence or other information may be done by researcher or diagnostician or may be done with the aid of a computer and databases as described below.

In another format, the levels of a gene(s) of Tables 5-5LL, its encoded protein(s), or any metabolite produced by the encoded protein may be monitored or detected in a Rankle, such as a bodily tissue or fluid sample to identify or diagnose a physiological state of an organism. Such samples may include any tissue or fluid sample, including urine, blood and easily obtainable cells such as peripheral lymphocytes.

Use of the Markers for Monitoring Toxicity Progression

As described above, the genes and gene expression information provided in Tables 5-5LL may also be used as markers for the monitoring of toxicity progression, such as that found after initial exposure to a drug, drug candidate, toxin, pollutant, etc. For instance, a tissue or cell sample may be assayed by any of the methods described above, and the expression levels from a gene or genes from Tables 5-5LL may be compared to the expression levels found in tissue or cells exposed to the cardiotoxins described herein. The comparison of the expression data, as well as available sequence or other information may be done by a researcher or diagnostician or may be done with the aid of a computer and databases.

Use of the Toxicity Markers for Drug Screening

According to the present invention, the genes identified in Tables 1-5LL may be used as markers or drug targets to evaluate the effects of a candidate drug, chemical compound or other agent on a cell or tissue sample. The genes may also be used as drug targets to screen for agents that modulate their expression and/or activity. In various formats, a candidate drug or agent can be screened for the ability to stimulate the transcription or expression of a given marker or markers or to down-regulate or counteract the transcription or expression of a marker or markers. According to the present invention, one can also compare the specificity of a drug's effects by looking at the number of markers which the drug induces and comparing them. More specific drugs will have less transcriptional targets. Similar sets of markers identified for two drugs may indicate a similarity of effects.

Assays to monitor the expression of a marker or markers as defined in Tables 1-5LL may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to one, two or more genes from Tables 1-5LL may be used to directly monitor or detect changes in gene expression in the treated or exposed cell. Cell lines, tissues or other samples are first exposed to a test agent and in some instances, a known toxin, and the detected expression levels of one or more, or preferably 2 or more of the genes of Tables 1-5LL are compared to the expression levels of those same genes exposed to a known toxin alone. Compounds that modulate the expression patterns of the known toxin(s) would be expected to modulate potential toxic physiological effects in vivo. The genes in Tables 1-5LL are particularly appropriate markers in these assays as they are differentially expressed in cells upon exposure to a known cardiotoxin. Tables 1 and 2 disclose those genes that are differentially expressed upon exposure to the named toxins and their corresponding GenBank Accession numbers. Table 3 discloses the human homologues and the corresponding GenBank Accession numbers of the differentially expressed genes of Tables 1 and 2.

In another format, cell lines that contain reporter gene fusions between the open reading frame and/or the transcriptional regulatory regions of a gene in Tables 1-5LL and any assayable fusion partner may be prepared. Numerous assayable fusion partners are known and readily available including the firefly luciferase gene and the gene encoding chloramphenicol acetyltransferase (Alam et al. (1990), Anal Biochem 188: 245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used to monitor the ability of the agent to modulate the expression of a gene identified in Tables 5-5LL. For instance, as described above, mRNA expression may be monitored directly by hybridization of probes to the nucleic acids of the invention. Cell lines are exposed to the agent to be tested under appropriate conditions and time, and total RNA or mRNA is isolated by standard procedures such those disclosed in Sambrook et al. (Molecular Cloning: A Laboratory Manual, 2nd Ed., Cold Spring Harbor Laboratory Press, Cold Spring Harbor, N.Y., 1989).

In another assay format, cells or cell lines are first identified which express the gene products of the invention physiologically. Cells and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines may be transduced or transfected with an expression vehicle (e.g., a plasmid or viral vector) construct comprising an operable non-translated 5′-promoter containing end of the structural gene encoding the gene products of Tables 1-5LL fused to one or more antigenic fragments or other detectable markers, which are peculiar to the instant gene products, wherein said fragments are under the transcriptional control of said promoter and are expressed as polypeptides whose molecular weight can be distinguished from the naturally occurring polypeptides or may further comprise an immunologically distinct or other detectable tag. Such a process is well known in the art (see Sambrook et al, supra).

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells are disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample is then compared with the control samples (no exposure and exposure to a known toxin) where only the excipient is contacted with the cells and an increase or decrease in the immunologically generated signal from the “agent-contacted” sample compared to the control is used to distinguish the effectiveness and/or toxic effects of the agent.

Use of Toxicity Markers to Identify Agents that Modulate Protein Activity or Levels

Another embodiment of the present invention provides methods for identifying agents that modulate at least one activity of a protein(s) encoded by the genes in Tables 1-5LL. Such methods or assays may utilize any means of monitoring or detecting the desired activity.

In one format, the relative amounts of a protein (Tables 1-5LL) between a cell population that has been exposed to the agent to be tested compared to an unexposed control cell population and a cell population exposed to a known toxin may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Agents that are assayed in the above methods can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” as used herein refers to the modification of a region or several regions of a peptide molecule to provide a structure chemically different from the parent peptide but topographically and functionally similar to the parent peptide (see G. A. Grant in: Molecular Biology and Biotechnology, Meyers, ed., pp. 659-664, VCH Publishers, New York, 1995). A skilled artisan can readily recognize that there is no limit as to the structural nature of the agents of the present invention.

Nucleic Acid Assay Formats

As previously discussed, the genes identified as being differentially expressed upon exposure to a known cardiotoxin (Tables 1-5LL) may be used in a variety of nucleic acid detection assays to detect or quantify the expression level of a gene or multiple genes in a given sample. The genes described in Tables 1-5LL may also be used in combination wits one or more additional genes whose differential expression is associate with toxicity in a cell or tissue. In preferred embodiments, the genes in Tables 5-5LL may be combined with one or more of the genes described in prior and related application 60/303,819 filed on Jul. 10, 2001; 60/305,623 filed on Jul. 17, 2001; 60/369,351 filed on Apr. 3, 2002; and 60/377,611 filed on May 6, 2002; Ser. Nos. 09/917,800; 10/060,087; 10/152,319; 10/191,803, and 10/301,856, all of which are incorporated by reference.

Any assay format to detect gene expression may be used. For example, traditional Northern blotting, dot or slot blot, nuclease protection, primer directed amplification, RT-PCR, semi- or quantitative PCR, branched-chain DNA and differential display methods may be used for detecting gene expression levels. Those methods are useful for some embodiments of the invention. In cases where smaller numbers of genes are detected, amplification based assays may be most efficient. Methods and assays of the invention, however, may be most efficiently designed with hybridization-based methods for detecting the expression of a large number of genes.

Any hybridization assay format may be used, including solution-based and solid support-based assay formats. Solid supports containing oligonucleotide probes for differentially expressed genes of the invention can be filters, polyvinyl chloride dishes, particles, beads, microparticles or silicon or glass based chips, etc. Such chips, wafers and hybridization methods are widely available, for example, those disclosed by Beattie (WO 95/11755).

Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000, 400,000 or 1,000,000 or more of such features on a single solid support. The solid support, or the area within which the probes are attached may be on the order of about a square centimeter. Probes corresponding to the genes of Tables 5-5LL or from the related applications described above may be attached to single or multiple solid support structures, e.g., the probes may be attached to a single chip or to multiple chips to comprise a chip set.

Oligonucleotide probe arrays for expression monitoring can be made and used according to any techniques known in the art (see for example, Lockhart et al. (1996), Nat Biotechnol 14:1675-1680; McGall et al. (1996), Proc Nat Acad Sci USA 93:13555-13460). Such probe arrays may contain at least two or more oligonucleotides that are complementary to or hybridize to two or more of the genes described in Tables 5-5LL. For instance, such arrays may contain oligonucleotides that are complementary to or hybridize to at least 2, 3, 4, 5, 6, 7, 8, 9, 10, 20, 30, 50, 70, 100 or more of the genes described herein. Preferred arrays contain all or nearly all of the genes listed in Tables 1-5LL, or individually, the gene sets of Tables 5-5LL. In a preferred embodiment, arrays are constructed that contain oligonucleotides to detect all or nearly all of the genes in any one of or all of Tables 1-5LL on a single solid support substrate, such as a chip.

The sequences of the expression marker genes of Tables 1-5LL are in the public databases. Table 1 provides the GenBank Accession Number for each of the sequences (see www.ncbi.nlm.nih.gov/) as well as a corresponding SEQ ID NO. in the sequence listing filed with this application. Table 3 provides the LocusLink and Unigene names and descriptions for the human homologues of the genes described in Tables 1 and 2. The sequences of the genes in GenBank and/or RefSeq are expressly herein incorporated by reference in their entirety as of the filing date of this application, as are related sequences, for instance, sequences from the same gene of different lengths, variant sequences, polymorphic sequences, genomic sequences of the genes and related sequences from different species, including the human counterparts, where appropriate (see Table 3). These sequences may be used in the methods of the invention or may be used to produce the probes and arrays of the invention. In some embodiments, the genes in Tables 1-5LL that correspond to the genes or fragments previously associated with a toxic response may be excluded from the Tables.

As described above, in addition to the sequences of the GenBank Accession Numbers disclosed in the Tables 1-5LL, sequences such as naturally occurring variants or polymorphic sequences may be used in the methods and compositions of the invention. For instance, expression levels of various allelic or homologous forms of a gene disclosed in Tables 1L-5LL may be assayed. Any and all nucleotide variations that do not significantly alter the functional activity of a gene listed in the Tables 1-5LL, including all naturally occurring allelic variants of the genes herein disclosed, may be used in the methods and to make the compositions (e.g., arrays) of the invention.

Probes based on the sequences of the genes described above may be prepared by any commonly available method. Oligonucleotide probes for screening or assaying a tissue of cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes described in Tables 1-5LL refer to oligonucleotides that are capable of hybridizing under stringent conditions to at least part of the nucleotide sequences of said genes, their encoded RNA or mRNA, or amplified versions of the RNA such as cRNA. Such hybridizable oligonucleotides will typically exhibit at least about 75% sequence identity at the nucleotide level to said genes, preferably about 80% or 85% sequence identity or more preferably about 90% or 95% or more sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids %. Background can also be calculated as the average signal intensity produced by regions of the array that lack any probes at all.

The phrase “hybridizing specifically to” or “specifically hybridizes” refers to the binding, duplexing, or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 100,000 or 1,000,000 or more different nucleic acid hybridizations.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases.

While the mismatch(es) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical submit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Probe Design

One of skill in the art will appreciate that an enormous number of array designs are suitable for the practice of this invention. The high density array will typically include a number of test probes that specifically hybridize to the sequences of interest. Probes may be produced from any region of the genes identified in the Tables and the attached representative sequence listing. In instances where the gene reference in the Tables is an EST, probes may be designed from that sequence or from other regions of the corresponding full-length transcript that may be available in any of the sequence databases, such as those herein described. See WO 99/32660 for methods of producing probes for a given gene or genes. In addition, any available software may be used to produce specific probe sequences, including, for instance, software available from Molecular Biology Insights, Olympus Optical Co. and Biosoft International. In a preferred embodiment, the array will also include one or more control probes.

High density array chips of the invention include “test probes.” Test probes may be oligonucleotides that range from about 5 to about 500, or about 7 to about 50 nucleotides, more preferably from about 10 to about 40 nucleotides and most preferably from about 15 to about 35 nucleotides in length. In other particularly preferred embodiments, the probes are 20 or 25 nucleotides in length. In another preferred embodiment, test probes are double or single strand DNA sequences such as cDNA fragments. DNA sequences are isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes may fall into three categories referred to herein as 1) normalization controls; 2) expression level controls; and 3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present the perfect match probes should be consistently brighter than the mismatch probes. In addition, if all central mismatches are present, the mismatch probes can be used to detect a mutation, for instance, a mutation of a gene in the accompanying Tables 1-5LL. The difference in intensity between the perfect match and the mismatch probe provides a good measure of the concentration of the hybridized material.

Nucleic Acid Samples

Cell or tissue samples may be exposed to the test agent in vitro or in vivo. When cultured cells or tissues are used, appropriate mammalian cell extracts, such as liver cell extracts, may also be added with the test agent to evaluate agents that may require biotransformation to exhibit toxicity.

The genes which are assayed according to the present invention are typically in the form of mRNA or reverse transcribed mRNA. The genes may or may not be cloned. The genes may or may not be amplified and cRNA produced. The cloning and/or amplification do not appear to bias the representation of genes within a population. In some assays, it may be preferable, however, to use polyA+ RNA as a source, as it can be used with less processing steps.

As is apparent to one of ordinary skill in the art, nucleic acid samples used in the methods and assays of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology. Vol. 24, Hybridization With Nucleic Acid Probes: Theory and Nucleic Acid Probes, P. Tijssen, Ed., Elsevier Press, New York, 1993. Such samples include RNA samples, but also include cDNA synthesized from a mRNA sample isolated from a cell or tissue of interest. Such samples also include DNA amplified from the cDNA, and RNA transcribed from the amplified DNA (cRNA). One of skill in the art would appreciate that it is desirable to inhibit or destroy RNase present in homogenates before homogenates are used.

Biological samples may be of any biological tissue or fluid or cells from any organism as well as cells raised in vitro, such as cell lines and tissue culture cells. Frequently the sample will be a tissue or cell sample that has been exposed to a compound, agent, drug, pharmaceutical composition, potential environmental pollutant or other composition. In some formats, the sample will be a “clinical sample” which is a sample derived from a patient. Typical clinical samples include, but are not limited to, sputum, blood, blood-cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

Forming High Density Arrays

Methods of forming nigh density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithographic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency, in this case in 6×SSPET at 37° C. (0.005% Triton X-100), to ensure hybridization and then subsequent washes are performed at higher stringency (e.g., 1×SSPET at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art. See WO 99/32660.

Databases

The present invention includes relational databases, such as the Gene Logic ToxExpress® database, containing sequence information, for instance, for the genes of Tables 1-5LL, as well as gene expression information from tissue or cells exposed to various standard toxins, such as those herein described (see Tables 5-5LL). Databases may also contain information associated with a given sequence or tissue sample such as descriptive information about the gene associated with the sequence information (see Tables 1 and 2), or descriptive information concerning the clinical status of the tissue sample, or the animal from which the sample was derived. The database may be designed to include different parts, for instance a sequence database and a gene expression database. Methods for the configuration and construction of such databases and computer-readable media to which such databases are saved are widely available, for instance, see U.S. Pat. No. 5,953,727, which is herein incorporated by reference in its entirety.

The databases of the invention may be linked to an outside or external database such as GenBank (www.ncbi.nlm.nih.gov/entrez.index.html); KEGG (www.genome.ad.jp/kegg); SPAD (www.grt.kyushu-u.acjp/spad/index.html); HUGO (www.gene.uclac.uk/hugo); Swiss-Prot (www.expasy.ch.sprot); Prosite (www.expasy.ch/tools/scnpsit1.html); OMI (www.ncbi.nlm.nih.gov/omim); and GDB (www.gdb.org). In a preferred embodiment, as described in Tables 1-3, the external database is GenBank and the associated databases maintained by the National Center for Biotechnology Information (NCBI) (www.ncbi.nlm.nih.gov).

Any appropriate computer platform, user interface, etc. may be used to perform the necessary comparisons between sequence information, gene expression information and any other information in the database or information provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, ToxScreen™ reports and electronic Northerns (E-NORTHERN™, Gene Logic, Inc., Gaithersburg, Md.) that allow the user to determine the cell type or tissue in which a given gene is expressed or allow determination of the abundance or expression level of a given gene in a particular tissue or cell, for instance, a cell or tissue sample exposed to a test compound.

The databases of the invention may also be used to present information identifying the expression level in a tissue or cell of a set of genes comprising one or more of the genes in Tables 5-5LL, comprising the step of comparing the expression level of at least one gene in Tables 5-5LL in a cell or tissue exposed to a test agent to the level of expression of the gene in the database. Such methods may be used to predict the toxic potential of a given compound by comparing the level of expression of a gene or genes in Tables 5-5LL from a tissue or cell sample exposed to the test agent to the expression levels found in a control tissue or cell samples exposed to a standard toxin or cardiotoxin such as those herein described. Such methods may also be used in the drug or agent screening assays as described herein.

Kits

The invention further includes kits combining, in different combinations, high-density oligonucleotide arrays, reagents for use with the arrays, protein reagents encoded by the genes of the Tables, signal detection and array-processing instruments, gene expression databases and analysis and database management software described above. The kits may be used, for example, to predict or model the toxic response of a test compound, to monitor the progression of heart disease states, to identify genes that show promise as new drug targets and to screen known and newly designed drugs as discussed above.

The databases packaged with the kits are a compilation of expression patterns from human or laboratory animal genes and gene fragments (corresponding to the genes of Tables 1-5LL). In particular, the database software and packaged information that may contain the databases saved to a computer-readable medium include the expression results of Tables 1-5LL that can be used to predict toxicity of a test agent by comparing the expression levels of the genes of Tables 1-5LL induced by the test agent to the expression levels presented in Tables 5-5LL. In another format, database and software information may be provided in a remote electronic format, such as a website, the address of which may be packaged in the kit.

Databases and software designed for use with microarrays is discussed in PCT/US99/20449, filed Sep. 8, 1999, Genomic Knowledge Discovery, PCT/IB00/00863, filed Jun. 28, 2000, Biological Data Processing, and in Balaban et al., U.S. Pat. No. 6,229,911, a computer-implemented method for managing information, stored as indexed tables, collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences.

The kits may be used in the pharmaceutical industry, where the need for early drug testing is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Identification of Toxicity Markers

The cardiotoxins adriamycin, amphotericin B, epirubicin, phenylpropanolamine, and rosiglitazone were administered to male Sprague-Dawley rats at various time points using administration diluents, protocols, and dosing regimes as indicated in Table 6. The cardiotoxins and control compositions, including cyclophosphamide, ifosfamide, minoxidil, hydralazine, BI-QT, clenbuterol, isoproterenol, norepinephrine, and epinephrine were administered to male Sprague-Dawley rats at various time points using administration diluents, protocols and dosing regimes as previously described in the art and previously described in the priority applications discussed above. The low and high dose level for each compound are provided in the chart below.

| Heart Toxin | Low Dose (mg/kg) | High Dose (mg/kg) |
|---|---|---|
| Cyclophosphamide | 20 | 200 |
| Ifosfamide | 5 | 100 |
| Minoxidil | 12 mg/L | 120 mg/L |
| Hydralazine | 2.5 | 25 |
| BI-QT | 10 | 50 |
| Clenbuterol | 0.4 | 4 |
| Isoproterenol | 0.05 | 0.5 |
| Norepinephrine | 0.05 | 0.5 |
| Epinephrine | 0.1 | 1 |
| Adriamycin | 1.3 | 12.8 |
| Amphotericin B | 0.25 | 2.5 |
| Epirubicin | 1.2 | 12 |
| Phenylpropanolamine | 6.4 | 64 |
| Rosiglitazone | 18 | 180 |

After administration, the dosed animals were observed and tissues were collected as described below:

Observation of Animals

1. Clinical Observations—Twice daily: mortality and moribundity check. Cage Side Observations—skin and fur, eyes and mucous membrane, respiratory system, circulatory system, autonomic and central nervous system, somatomotor pattern, and behavior pattern. Potential signs of toxicity, including tremors, convulsions, salivation, diarrhea, lethargy, coma or other atypical behavior or appearance, were recorded as they occurred and included a time of onset, degree, and duration.

2. Physical Examinations—Prior to randomization, prior to initial treatment, and prior to sacrifice.

3. Body Weights—Prior to randomization, prior to initial treatment, and prior to sacrifice.

Clinical Pathology

1. Frequency Prior to necropsy.

2. Number of animals All surviving animals.

3. Bleeding Procedure Blood was obtained by puncture of the orbital sinus while under 70% CO₂/30% O₂ anesthesia.

4. Collection of Blood Samples Approximately 0.5 mL of blood was collected into EDTA tubes for evaluation of hematology parameters. Approximately 1 mL of blood was collected into serum separator tubes for clinical chemistry analysis. Approximately 200 μL of plasma was obtained and frozen at ˜−80° C. for test compound/metabolite estimation. An additional ˜2 mL of blood was collected into a 15 mL conical polypropylene vial to which ˜3 mL of Trizol was immediately added. The contents were immediately mixed with a vortex and by repeated inversion. The tubes were frozen liquid nitrogen and stored at ˜−80° C.

Termination Procedures

Terminal Sacrifice

At the sampling times indicated in Tables 5A-5LL and Table 6 for each cardiotoxin, and as previously described in the related applications mentioned above, rats were weighed, physically examined, sacrificed by decapitation, and exsanguinated. The animals were necropsied within approximately five minutes of sacrifice. Separate sterile, disposable instruments were used for each animal, with the exception of bone cutters, which were used to open the skull cap. The bone cutters were dipped in disinfectant solution between animals.

Necropsies were conducted on each animal following procedures approved by board-certified pathologists.

Animals not surviving until terminal sacrifice were discarded without necropsy (following euthanasia by carbon dioxide asphyxiation, if moribund). The approximate time of death for moribund or found dead animals was recorded.

Postmortem Procedures

Fresh and sterile disposable instruments were used to collect tissues. Gloves were worn at all times when handling tissues or vials. All tissues were collected and frozen within approximately 5 minutes of the animal's death. The liver sections and kidneys were frozen within approximately 3-5 minutes of the animal's death. The time of euthanasia, an interim time point at freezing of liver sections and kidneys, and time at completion of necropsy were recorded. Tissues were stored at approximately −80° C. or preserved in 10% neutral buffered formalin.

Tissue Collection and Processing

Liver—

1. Right medial lobe—snap frozen in liquid nitrogen and stored at −80° C.

2. Left medial lobe—Preserved in 10% neutral-buffered formalin (NBF) and evaluated for gross and microscopic pathology.

3. Left lateral lobe—snap frozen in liquid nitrogen and stored at ˜−80° C.

Heart—

A sagittal cross-section containing portions of the two atria and of the two ventricles was preserved in 10% NBF. The remaining heart was frozen in liquid nitrogen and stored at ˜−80° C.

Kidneys (Both)—

1. Left—Hemi-dissected; half was preserved in 10% NBF and the remaining half was frozen in liquid nitrogen and stored at ˜−80° C.

2. Right—Hemi-dissected; half was preserved in 10% NBF and the remaining half was frozen in liquid nitrogen and stored at ˜−80° C.

Testes (Both)—

A sagittal cross-section of each testis was preserved in 10% NBF. The remaining testes were frozen together in liquid nitrogen and stored at −80° C.

Brain (Whole)—

A cross-section of the cerebral hemispheres and of the diencephalon was preserved in 10% NBF, and the rest of the brain was frozen in liquid nitrogen and stored at ˜−80° C.

Microarray sample preparation was conducted with minor modifications, following the protocols set forth in the Affymetrix GeneChip Expression Analysis Manual. Frozen tissue was ground to a powder using a Spex Certiprep 6800 Freezer Mill. Total RNA was extracted with Trizol (GibcoBRL) utilizing the manufacturer's protocol. The total RNA yield for each sample was 200-500 μg per 300 mg tissue weight. mRNA was isolated using the Oligotex mRNA Midi kit (Qiagen) followed by ethanol precipitation. Double stranded cDNA was generated from mRNA using the SuperScript Choice system (GibcoBRL). First strand cDNA synthesis was primed with a T7-(dT24) oligonucleotide. The cDNA was phenol-chloroform extracted and ethanol precipitated to a final concentration of 1 μg/ml. From 2 μg of cDNA, cRNA was synthesized using Ambion's T7 MegaScript in vitro Transcription Kit.

To biotin label the cRNA, nucleotides Bio-11-CTP and Bio-16-UTP (Enzo Diagnostics) were added to the reaction. Following a 37° C. incubation for six hours, impurities were removed from the labeled cRNA following the RNeasy Mini kit protocol (Qiagen). cRNA was fragmented (fragmentation buffer consisting of 200 mM Tris-acetate, pH 8.1, 500 mM KOAc, 150 mM MgOAc) for thirty-five minutes at 94° C. Following the Affymetrix protocol, 55 μg of fragmented cRNA was hybridized on the Affymetrix rat array set for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Data was analyzed using Affymetrix GeneChip® version 2.0 and Expression Data Mining (EDMT) software (version 1.0), Gene Logic's GeneExpress® 2000 software and S-Plus™ software.

Tables 1 and 2 disclose those genes that are differentially expressed upon exposure to the named toxins and their corresponding GenBank Accession and Sequence Identification numbers, the identities of the metabolic pathways in which the genes function, the gene names if known, and the Unigene Cluster titles. The human homologues of the rat genes in Tables 1 and 2 are indicated in Table 3. The model codes in Tables 1-3 represent the various toxicity or heart pathology states that differential expression of each gene is able to identify, as well as the individual toxin or toxin type associated with differential expression of each gene. The model codes are defined in Table 4. The GLGC ID is the internal Gene Logic identification number.

Tables 5A-5LL disclose a set of genes, along with the summary statistics for each of the comparisons performed as indicated in these tables, i.e., expression levels of a particular gene in toxicity group samples compared to non-toxicity group samples in response to exposure to a particular toxin, or as measured in a particular disease state. Each of these tables contains a set of predictive genes and creates a model for predicting the cardiotoxicity of an unknown, i.e., untested compound. Each gene is identified by its Gene Logic identification number and can be cross-referenced to a gene name and representative sequence identification number in Tables 1 and 2 or in one or more related applications, as mentioned on page 1.

For each comparison of gene expression levels between samples in the toxicity group (samples affected by exposure to a specific toxin) and samples in the non-toxicity group (samples not affected by exposure to that same specific toxin), the tox mean (for toxicity group samples) is the mean signal intensity, as normalized for the various chip parameters that are being assayed. The non-tox mean represents the mean signal intensity, as normalized for the various chip parameters that are being assayed, in samples from animals other than those treated with the high dose of the specific toxin. These animals were treated with a low dose of the specific toxin, or with vehicle alone, or with a different toxin. Samples in the toxicity groups were obtained from animals sacrificed at the time point(s) indicated in the Table 5-5LL headings, while samples in the non-toxicity groups were obtained from animals sacrificed at all time points in the experiments. For individual genes, an increase in the tox mean compared to the non-tox mean indicates up-regulation upon exposure to a toxin. Conversely, a decrease in the tox mean compared to the non-tox mean indicates down-regulation.

The mean values are derived from Average Difference (AveDiff) values for a particular gene, averaged across the corresponding samples. Each individual Average Difference value is calculated by integrating the intensity information from multiple probe pairs that are tiled for a particular fragment. The normalization multiplies each expression intensity for a given experiment (chip) by a global scaling factor. The intent of this normalization is to make comparisons of individual genes between chips possible. The scaling factor is calculated as follows:

1. From all the unnormalized expression values in the experiment, delete the largest 2% and smallest 2% of the values. That is, if the experiment yields 10,000 expression values, order the values and delete the smallest 200 and largest 200.

2. Compute the trimmed mean, which is equal to the mean of the remaining values.

3. Compute the scale factor SF=100/(trimmed mean)

Values greater than 2.0*SD noise are assumed to come from expressers. For these values, the standard deviation SD log (signal) of the logarithms is calculated. The logarithms are then multiplied by a scale factor proportional to 1/SD log (signal) and exponentiated. The resulting values are then multiplied by another scale factor, chosen so there will be no discontinuity in the normalized values from unscaled values on either side of 2.0*SD noise. Some AveDiff values may be negative due to the general noise involved in nucleic acid hybridization experiments. Although many conclusions can be made corresponding to a negative value on the GeneChip platform, it is difficult to assess the meaning behind the negative value for individual fragments. Our observations show that, although negative values are observed at times within the predictive gene set, these values reflect a real biological phenomenon that is highly reproducible across all the samples from which the measurement was taken. For this reason, those genes that exhibit a negative value are included in the predictive set. It should be noted that other platforms of gene expression measurement may be able to resolve the negative numbers for the corresponding genes. The predictive ability of each of those genes should extend across platforms, however. Each mean value is accompanied by the standard deviation for the mean. The linear discriminant analysis score (discriminant score, or LDA), as disclosed in the tables, measures the ability of each gene to predict whether or not a sample is toxic. The discriminant score is calculated by the following steps:

Calculation of a Discriminant Score

Let Xᵢ represent the AveDiff values for a given gene across the non-tox samples, i=1 . . . n.

Let Yᵢ represent the AveDiff values for a given gene across the tox samples, i=1 . . . t.

The calculations proceed as follows:

1. Calculate mean and standard deviation for Xᵢ's and Yᵢ's, and denote these by m_{X}, m_{Y}, s_{X},s_{Y}.

2. For all Xᵢ's and Yᵢ's, evaluate the function f(z)=((1/s_{Y})*exp(−0.5*((z−m_{Y})/s_{Y})²))/(((1/s_{Y})*exp(−0.5*((z−m_{Y})/s_{Y})²))+((1/s_{X})*exp(−0.5*((z−m_{X})/s_{X})²))).

3. The number of correct predictions, say P, is then the number of Yᵢ's such that f(Yᵢ)>0.5 plus the number of Xᵢ's such that f(Xᵢ)<0.5.

4. The discriminant score is then P/(n+t).

Linear discriminant analysis uses both the individual measurements of each gene and the calculated measurements of all combinations of genes to classify samples. For each gene a weight is derived from the mean and standard deviation of the toxic and nontox groups. Every gene is multiplied by a weight and the sum of these values results in a collective discriminate score. This discriminant score is then compared against collective centroids of the tox and nontox groups. These centroids are the average of all tox and nontox samples respectively. Therefore, each gene contributes to the overall prediction. This contribution is dependent on weights that are large positive or negative numbers if the relative distances between the tox and nontox samples for that gene are large and small numbers if the relative distances are small. The discriminant score for each unknown sample and centroid values can be used to calculate a probability between zero and one as to the group in which the unknown sample belongs.

### Example 2

### General Tonicity Modeling

Samples were selected for grouping into tox-responding and non-tox-responding groups by examining each study individually with Principal Components Analysis (PCA) to determine which treatments had an observable response. Only groups where confidence of their tox-responding and non-tox-responding status was established were included in building a general tox model (Tables 5A-5LL).

Linear discriminant models were generated to describe toxic and non-toxic samples. The top discriminant genes and/or EST's were used to determine toxicity by calculating each gene's contribution with homo and heteroscedastic treatment of variance and inclusion or exclusion of mutual information between genes. Prediction of samples within the database exceeded 80% true positives with a false positive rate of less than 5%. It was determined that combinations of genes and/or EST's generally provided a better predictive ability than individual genes and that the more genes and/or EST used the better predictive ability. Although the preferred embodiment includes fifty or more genes, many pairings or greater combinations of genes and/or EST can work better than individual genes. All combinations of two or more genes from the selected list (Tables 5A-5LL) could be used to predict toxicity. These combinations could be selected by pairing in an agglomerate, divisive, or random approach. Further, as yet undetermined genes and/or EST's could be combined with individual or combination of genes and/or EST's described here to increase predictive ability. However, the genes and/or EST's described here would contribute most of the predictive ability of any such undetermined combinations.

Other variations on the above method can provide adequate predictive ability. These include selective inclusion of components via agglomerate, divisive, or random approaches or extraction of loading and combining them in agglomerate, divisive, or random approaches. Also the use of composite variables in logistic regression to determine classification of samples can also be accomplished with linear discriminate analysis, neural or Bayesian networks, or other forms of regression and classification based on categorical or continual dependent and independent variables.

### Example 3

### Modeling with Core Gene Set

As described in Examples 1 and 2, above, the data collected from microarray hybridization experiments were analyzed by LDA and by PCA. The genes in Tables 5G, 5I, 5K, 5M, 5O, 5Q, 5T, 5V, 5X, 5Z, 5BB, 5DD, 5FF, and 5KK constitute a core set of markers for predicting the cardiotoxicity of a compound, whereas the genes in Tables 5H, 5I, 5L, 5N, 5P, 5S, 5U, 5W, 5Y, 5AA, 5CC and 5EE constitute an alternative set of markers. The core marker tables comprise genes that are also found in PCT Application No. PCT/US02/2735, whereas the alternate marker tables do not comprise genes also found in the '735 application. Each gene fragment in Tables 1-5LL is assigned an LDA score, and those gene fragments in the core set are those with the highest LDA scores. The gene fragments in Tables 5A-5LL were determined to give greater than 80% true positive results and less than 5% false positive results. Gene expression profiles prepared or obtained from expression data for these genes, in the presence and absence of toxin treatment, can be used a controls in assays of compounds whose toxic properties have not been examined. Comparison of data from test compound-exposed and test compound-unexposed animals with the data in Tables 5A-5LL allows the prediction of toxic effects—or no toxic effects—upon exposure to the test compound. Thus, the marker gene sets can be used to examine the biological effects of a compound whose toxic properties following exposure are not known and to predict the toxicity in cardiac tissue of this compound.

### Example 4

### Modeling Methods

The above modeling methods provide broad approaches of combining the expression of genes to predict sample toxicity. One could also provide no weight in a simple voting method or determine weights in a supervised or unsupervised method using agglomerate, divisive, or random approaches. All or selected combinations of genes may be combined in ordered, agglomerate, or divisive, supervised or unsupervised clustering algorithms with unknown samples for classification. Any form of correlation matrix may also be used to classify unknown samples. The spread of the group distribution and discriminate score alone provide enough information to enable a skilled person to generate all of the above types of models with accuracy that can exceed discriminate ability of individual genes. Some examples of methods that could be used individually or in combination after transformation of data types include but are not limited to: Discriminant Analysis, Multiple Discriminant Analysis, robust multi-array average (RMA) analysis, partial least squares (PLS) analysis, logistic regression, multiple regression analysis, linear regression analysis, conjoint analysis, canonical correlation, hierarchical cluster analysis, k-means cluster analysis, self-organizing maps, multidimensional scaling, structural equation modeling, support vector machine determined boundaries, factor analysis, neural networks, bayesian classifications, and resampling methods.

### Example 5

### Individual Compound Markers

Samples were grouped into individual pathology classes based on known toxicological responses and observed clinical chemical and pathology measurements or into early and late phases of observable toxicity within a compound (Tables 1-5LL). The top 10, 25, 50, 100 genes based on individual discriminate scores were used in a model to ensure that combination of genes provided a better prediction than individual genes. As described above, all combinations of two or more genes from this list could potentially provide better prediction than individual genes when selected in any order or by ordered, agglomerate, divisive, or random approaches. In addition, combining these genes with other genes could provide better predictive ability, but most of this predictive ability would come from the genes listed herein.

Samples may be considered toxic if they score positive in any individual compound represented here or in any modeling method mentioned under general toxicology models based on combination of individual time and dose grouping of individual toxic compounds obtainable from the data. Most logical groupings with one or more genes and one or more sample dose and time points should produce better predictions of general toxicity or similarity to known toxicant than individual genes.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

LENGTHY TABLE REFERENCED HERE
US20070054269A1-20070308-T00001
Please refer to the end of the specification for access instructions.

LENGTHY TABLE REFERENCED HERE
US20070054269A1-20070308-T00002
Please refer to the end of the specification for access instructions.

LENGTHY TABLE REFERENCED HERE
US20070054269A1-20070308-T00003
Please refer to the end of the specification for access instructions.

LENGTHY TABLE REFERENCED HERE
US20070054269A1-20070308-T00004
Please refer to the end of the specification for access instructions.

LENGTHY TABLE REFERENCED HERE
US20070054269A1-20070308-T00005
Please refer to the end of the specification for access instructions.

LENGTHY TABLE REFERENCED HERE
US20070054269A1-20070308-T00006
Please refer to the end of the specification for access instructions.

LENGTHY TABLE REFERENCED HERE
US20070054269A1-20070308-T00007
Please refer to the end of the specification for access instructions.

LENGTHY TABLE REFERENCED HERE
US20070054269A1-20070308-T00008
Please refer to the end of the specification for access instructions.

LENGTHY TABLE REFERENCED HERE
US20070054269A1-20070308-T00009
Please refer to the end of the specification for access instructions.

LENGTHY TABLE REFERENCED HERE
US20070054269A1-20070308-T00010
Please refer to the end of the specification for access instructions.

LENGTHY TABLE REFERENCED HERE
US20070054269A1-20070308-T00011
Please refer to the end of the specification for access instructions.

LENGTHY TABLE REFERENCED HERE
US20070054269A1-20070308-T00012
Please refer to the end of the specification for access instructions.

LENGTHY TABLE REFERENCED HERE
US20070054269A1-20070308-T00013
Please refer to the end of the specification for access instructions.

LENGTHY TABLE REFERENCED HERE
US20070054269A1-20070308-T00014
Please refer to the end of the specification for access instructions.

LENGTHY TABLE REFERENCED HERE
US20070054269A1-20070308-T00015
Please refer to the end of the specification for access instructions.

LENGTHY TABLE REFERENCED HERE
US20070054269A1-20070308-T00016
Please refer to the end of the specification for access instructions.

LENGTHY TABLE REFERENCED HERE
US20070054269A1-20070308-T00017
Please refer to the end of the specification for access instructions.

LENGTHY TABLE REFERENCED HERE
US20070054269A1-20070308-T00018
Please refer to the end of the specification for access instructions.

LENGTHY TABLE REFERENCED HERE
US20070054269A1-20070308-T00019
Please refer to the end of the specification for access instructions.

LENGTHY TABLE REFERENCED HERE
US20070054269A1-20070308-T00020
Please refer to the end of the specification for access instructions.

LENGTHY TABLE REFERENCED HERE
US20070054269A1-20070308-T00021
Please refer to the end of the specification for access instructions.

LENGTHY TABLE REFERENCED HERE
US20070054269A1-20070308-T00022
Please refer to the end of the specification for access instructions.

LENGTHY TABLE REFERENCED HERE
US20070054269A1-20070308-T00023
Please refer to the end of the specification for access instructions.

LENGTHY TABLE REFERENCED HERE
US20070054269A1-20070308-T00024
Please refer to the end of the specification for access instructions.

LENGTHY TABLE REFERENCED HERE
US20070054269A1-20070308-T00025
Please refer to the end of the specification for access instructions.

LENGTHY TABLE REFERENCED HERE
US20070054269A1-20070308-T00026
Please refer to the end of the specification for access instructions.

LENGTHY TABLE REFERENCED HERE
US20070054269A1-20070308-T00027
Please refer to the end of the specification for access instructions.

LENGTHY TABLE REFERENCED HERE
US20070054269A1-20070308-T00028
Please refer to the end of the specification for access instructions.

LENGTHY TABLE REFERENCED HERE
US20070054269A1-20070308-T00029
Please refer to the end of the specification for access instructions.

LENGTHY TABLE REFERENCED HERE
US20070054269A1-20070308-T00030
Please refer to the end of the specification for access instructions.

LENGTHY TABLE REFERENCED HERE
US20070054269A1-20070308-T00031
Please refer to the end of the specification for access instructions.

LENGTHY TABLE REFERENCED HERE
US20070054269A1-20070308-T00032
Please refer to the end of the specification for access instructions.

LENGTHY TABLE REFERENCED HERE
US20070054269A1-20070308-T00033
Please refer to the end of the specification for access instructions.

LENGTHY TABLE REFERENCED HERE
US20070054269A1-20070308-T00034
Please refer to the end of the specification for access instructions.

LENGTHY TABLE REFERENCED HERE
US20070054269A1-20070308-T00035
Please refer to the end of the specification for access instructions.

LENGTHY TABLE REFERENCED HERE
US20070054269A1-20070308-T00036
Please refer to the end of the specification for access instructions.

LENGTHY TABLE REFERENCED HERE
US20070054269A1-20070308-T00037
Please refer to the end of the specification for access instructions.

LENGTHY TABLE REFERENCED HERE
US20070054269A1-20070308-T00038
Please refer to the end of the specification for access instructions.

LENGTHY TABLE REFERENCED HERE
US20070054269A1-20070308-T00039
Please refer to the end of the specification for access instructions.

LENGTHY TABLE REFERENCED HERE
US20070054269A1-20070308-T00040
Please refer to the end of the specification for access instructions.

LENGTHY TABLE REFERENCED HERE
US20070054269A1-20070308-T00041
Please refer to the end of the specification for access instructions.

LENGTHY TABLE REFERENCED HERE
US20070054269A1-20070308-T00042
Please refer to the end of the specification for access instructions.

LENGTHY TABLE REFERENCED HERE
US20070054269A1-20070308-T00043
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070054269A1). An electronic copy of the table will also be available from the USPTO upon request and payment of the fee set forth in 37 CFR 1.19(b)(3).

1. A method of predicting at least one toxic effect of a compound, comprising:
(a) obtaining a gene expression profile of a tissue or cell sample exposed to the compound; and
(b) comparing the gene expression profile to a database comprising substantially all of the data or information of Tables 5A-5LL.

2. A method of claim 1, wherein the gene expression profile obtained from the tissue or cell sample comprises the level of expression for at least one gene.

3. A method of claim 2, wherein the level of expression is compared to a Tox Mean and/or NonTox Mean value in Tables 5A-5LL.

4. A method of claim 3, wherein the level of expression is normalized prior to comparison.

5. A method of claim 1, wherein the database comprises substantially all of the data or information in Tables 5A-5LL.

6. A method of claim 1, wherein the tissue or cell sample is a heart tissue or heart cell sample.

7. A method of predicting at least one toxic effect of a compound, comprising:
(a) detecting the level of expression in a tissue or cell sample exposed to the compound of two or more genes from Tables 5C, 5D, 5E, 5F, 5H, 5J, 5L, 5N, 5P, 5R, 5S, 5U, 5W, 5Y, 5AA, 5CC, 5EE, 5GG, 5HH, and 5II; wherein differential expression of the genes in Tables 5C, 5D, 5E, 5F, 5H, 5J, 5L, 5N, 5P, 5R, 5S, 5U, 5W, 5Y, 5AA, 5CC, 5EE, 5GG, 5HH, and 5II is indicative of at least one toxic effect.

8. A method of predicting the progression of a toxic effect of a compound, comprising:
(a) detecting the level of expression in a tissue or cell sample exposed to the compound of two or more genes from Tables 5C, 5D, 5E, 5F, 5H, 5J, 5L, 5N, 5P, 5R, 5S, 5U, 5W, 5Y, 5AA, 5CC, 5EE, 5GG, 5HH, and 5II; wherein differential expression of the genes in Tables 5C, 5D, 5E, 5F, 5H, 5J, 5L, 5N, 5P, 5R, 5S, 5U, 5W, 5Y, 5AA, 5CC, 5EE, 5GG, 5HH, and 5II is indicative of toxicity progression.

9. A method of predicting the cardiotoxicity of a compound, comprising:
(a) detecting the level of expression in a tissue or cell sample exposed to the compound of two or more genes from 5C, 5D, 5E, 5F, 5H, 5J, 5L, 5N, 5P, 5R, 5S, 5U, 5W, 5Y, 5AA, 5CC, 5EE, 5GG, 5HH, and 5II; wherein differential expression of the genes in 5C, 5D, 5E, 5F, 5H, 5J, 5L, 5N, 5P, 5R, 5S, 5U, 5W, 5Y, 5AA, 5CC, 5EE, 5GG, 5HH, and 5II is indicative of cardiotoxicity.

10. A method of identifying an agent that modulates the onset or progression of a toxic response, comprising:
(a) exposing a cell to the agent and a known toxin; and
(b) detecting the agent induced change in the expression level of two or more genes from Tables 5C, 5D, 5E, 5F, 5H, 5J, 5L, 5N, 5P, 5R, 5S, 5U, 5W, 5Y, 5AA, 5CC, 5EE, 5GG, 5HH, and 5II; wherein differential expression of the genes in Tables 5C, 5D, 5E, 5F, 5H, 5J, 5L, 5N, 5P, 5R, 5S, 5U, 5W, 5Y, 5AA, 5CC, 5EE, 5GG, 5HH, and 5II, compared to a control, is indicative of toxicity.

11. A method of predicting the cellular pathways that a compound modulates in a cell, comprising:
(a) detecting the level of expression in a tissue or cell sample exposed to the compound of two or more genes from Tables 5C, 5D, 5E, 5F, 5H, 5J, 5L, 5N, 5P, 5R, 5S, 5U, 5W, 5Y, 5AA, 5CC, 5EE, 5GG, 5HH, and 5II; wherein differential expression of the genes in Tables 5C, 5D, 5E, 5F, 5H, 5J, 5L, 5N, 5P, 5R, 5S, 5U, 5W, 5Y, 5AA, 5CC, 5EE, 5GG, 5HH, and 5II is associated the modulation of at least one cellular pathway.

12. The method of claim 7, wherein the expression levels of at least 5 genes are detected.

13. The method of claim 7, wherein the expression levels of at least 10 genes are detected.

14. The method of claim 7, wherein the expression levels of at least 25 genes are detected.

15. The method of claim 7, wherein the expression levels of at least 50 genes are detected.

16. The method of claim 7, wherein the expression levels of at least 100 genes are detected.

17. The method of claim 7, wherein the expression levels of at least 200 genes are detected.

18. The method of claim 7, wherein the expression levels of at least 500 genes are detected.

19. The method of claim 7, wherein the expression levels of nearly all genes are detected.

20. A method of claim 7, wherein the effect is selected from the group consisting of myocarditis, arrhythmias, tachycardia, myocardial ischemia, angina, hypertension, hypotension, dyspnea, and cardiogenic shock.

21. A method of claim 9, wherein the cardiotoxicity is associated with at least one heart disease pathology selected from the group consisting of myocarditis, arrhythmias, tachycardia, myocardial ischemia, angina, hypertension, hypotension, dyspnea, and cardiogenic shock.

22. A method of claim 11, wherein the cellular pathway is modulated by a toxin selected from the group consisting of cyclophosphamide, ifosfamide, minoxidil, hydralazine, BI-QT, clenbuterol, isoproterenol, norepinephrine, epinephrine, adriamycin, amphotericin B, epirubicin, phenylpropanolamine, and rosiglitazone.

23. A set of at least two probes, wherein each of the probes comprises a sequence that specifically hybridizes to a gene in Tables 5C, 5D, 5E, 5F, 5H, 5J, 5L, 5N, 5P, 5R, 5S, 5U, 5W, 5Y, 5AA, 5CC, 5EE, 5GG, 5HH, and 5II.

24. A set of probes according to claim 23, wherein the set comprises probes that hybridize to at least 10 genes.

25. A set of probes according to claim 23, wherein the set comprises probes that hybridize to at least 50 genes.

26. A set of probes according to claim 23, wherein the set comprises probes that hybridize to at least 100 genes.

27. A set of probes according to claim 23, wherein the set comprises probes that hybridize to at least 500 genes.

28. A set of probes according to claim 23, wherein the probes are attached to a solid support.

29. A set of probes according to claim 28, wherein the solid support is selected from the group consisting of a membrane, a glass support and a silicon support.

30. A solid support comprising at least two probes, wherein each of the probes comprises a sequence that specifically hybridizes to a gene in Tables 5C, 5D, 5E, 5F, 5H, 5J, 5L, 5N, 5P, 5R, 5S, 5U, 5W, 5Y, 5AA, 5CC, 5EE, 5GG, 5HH, and 5II.

31. A solid support of claim 30, wherein the solid support is an array comprising at least 10 different oligonucleotides in discrete locations per square centimeter.

32. A solid support of claim 31, wherein the array comprises at least about 100 different oligonucleotides in discrete locations per square centimeter.

33. A solid support of claim 31, wherein the array comprises at least about 1000 different oligonucleotides in discrete locations per square centimeter.

34. A solid support of claim 31, wherein the array comprises at least about 10,000 different oligonucleotides in discrete locations per square centimeter.

35. A computer system comprising:
(a) a database containing information identifying the expression level in a tissue or cell sample exposed to a cardiotoxin of a set of genes comprising at least two genes in Tables 5C, 5D, 5E, 5F, 5H, 5J, 5L, 5N, 5P, 5R, 5S, 5U, 5W, 5Y, 5AA, 5CC, 5EE, 5GG, 5HH, and 5II; and
(b) a user interface to view the information.

36. A computer system of claim 35, wherein the database further comprises sequence information for the genes.

37. A computer system of claim 35, wherein the database further comprises information identifying the expression level for the set of genes in the tissue or cell sample before exposure to a cardiotoxin.

38. A computer system of claim 35, wherein the database further comprises information identifying the expression level of the set of genes in a tissue or cell sample exposed to at least a second cardiotoxin.

39. A computer system of claim 35, further comprising records including descriptive information from an external database, which information correlates said genes to records in the external database.

40. A computer system of claim 39, wherein the external database is GenBank.

41. A method of using a computer system of claim 35 to present information identifying the expression level in a tissue or cell of at least one gene in Tables 5A-5LL, comprising:
comparing the expression level of at least one gene in Tables 5A-5LL in a tissue or cell exposed to a test agent to the level of expression of the gene in the database.

42. A method of claim 41, wherein the expression levels of at least 10 genes are compared.

43. A method of claim 41, wherein the expression levels of at least 100 genes are compared.

44. A method of claim 41, wherein the expression levels of at least 500 genes are compared.

45. A method of claim 41, further comprising the step of displaying the level of expression of at least one gene in the tissue or cell sample compared to the expression level when exposed to a toxin.

46. A method of claim 10, wherein the known toxin is a cardiotoxin.

47. A method of claim 43, wherein the cardiotoxin is selected from the group consisting of cyclophosphamide, ifosfamide, minoxidil, hydralazine, BI-QT, clenbuterol, isoproterenol, norepinephrine, epinephrine, adriamycin, amphotericin B, epirubicin, phenylpropanolamine, and rosiglitazone.

48. A method of claim 7, wherein nearly all of the genes in Tables 5A-5LL are detected.

49. A method of claim 48, wherein all of the genes in at least one of Tables 5C, 5D, 5E, 5F, 5H, 5J, 5L, 5N, 5P, 5R, 5S, 5U, 5W, 5Y, 5AA, 5CC, 5EE, 5GG, 5HH, and 5II are detected.

50. A kit comprising at least one solid support of claim 30 packaged with gene expression information for said genes.

51. A kit of claim 50, wherein the gene expression information comprises gene expression levels in a tissue or cell sample exposed to a cardiotoxin.

52. A kit of claim 51, wherein the gene expression information is in an electronic format.

53. A method of claim 7, wherein the compound exposure is in vivo or in vitro.

54. A method of claim 7, wherein the level of expression is detected by an amplification or hybridization assay.

55. A method of claim 54, wherein the amplification assay is quantitative or semi-quantitative PCR.

56. A method of claim 54, wherein the hybridization assay is selected from the group consisting of Northern blot, dot or slot blot, nuclease protection and microarray assays.

57. A method of identifying an agent that modulates at least one activity of a protein encoded by a gene in Tables 5C, 5D, 5E, 5F, 5H, 5J, 5L, 5N, 5P, 5R, 5S, 5U, 5W, 5Y, 5AA, 5CC, 5EE, 5GG, 5HH, and 5II comprising:
(a) exposing the protein to the agent; and
(b) assaying at least one activity of said protein.

58. A method of claim 57, wherein the agent is exposed to a cell expressing the protein.

59. A method of claim 58, wherein the cell is exposed to a known toxin.

60. A method of claim 59 wherein the toxin modulates the expression of the protein.

61. A method of predicting at least one toxic effect of a compound, comprising:
(a) obtaining a gene expression profile of a tissue or cell sample exposed to the compound; and
(b) comparing the gene expression profile to a database comprising at least part of the data or information of Tables 5A, 5B, 5G, 5I, 5K, 5M, 5O, 5Q, 5T, 5V, 5X, 5Z, 5BB, 5DD, 5FF, 5JJ, 5KK, and 5LL.

62. A method of claim 61, wherein the gene expression profile obtained from the tissue or cell sample comprises the level of expression for at least one gene.

63. A method of claim 62, wherein the level of expression is compared to a Tox Mean and/or Non-Tox Mean value in Tables 5A, 5B, 5G, 5I, 5K, 5M, 5O, 5Q, 5T, 5V, 5X, 5Z, 5BB, 5DD, 5FF, 5JJ, 5KK, and 5LL.

64. A method of claim 63, wherein the level of expression is normalized prior to comparison.

65. A method of claim 61, wherein the database comprises substantially all of the data or information in Tables 5A, 5B, 5G, 5I, 5K, 5M, 5O, 5Q, 5T, 5V, 5X, 5Z, 5BB, 5DD, 5FF, 5JJ, 5KK, and 5LL.

66. A method of claim 61, wherein the tissue or cell sample is a heart tissue or heart cell sample.
